(19) United States
(12) Patent Application Publication
(10) Pub. No.: US 2017/0143786 A1
(43) Pub. Date: May 25, 2017

(54) INTRAVAGINAL FORMULATIONS COMPRISING GnRH

(71) Applicant: Zoetis Services LLC, Parsippany, NJ (US)

(72) Inventors: Kenneth L. Feenstra, Kalamazoo, MI (US); Laibin Luo, Kalamazoo, MI (US); Susan C. Sucheta, Kalamazoo, MI (US); Nicholas A. Wicks, Kalamazoo, MI (US); Karen P. Kolbasa, Kalamazoo, MI (US)

(21) Appl. No.: 15/318,851

(22) PCT Filed: Jun. 3, 2015
(86) PCT No.: PCT/US2015/033951

§ 371 Date: Dec. 14, 2016

Related U.S. Application Data
(60) Provisional application No. 62/012,523, filed on Jun. 16, 2014.

(51) Int. Cl. A61K 38/09; A61K 9/00; A61K 47/44; A61K 47/12

(52) U.S. Cl. CPC A61K 38/09; A61K 47/12; A61K 9/0034; A61K 47/44

(57) ABSTRACT

An intravaginal formulation is provided, comprising a carboxylic acid in a n amount of 10-20% w/w of said formulation, an oleaginous base, having melting temperature between 30° C. and 38° C., GnRH or an analog thereof, in an amount equivalent to GnRH amount of between 10 μg and 600 μg. A method of inducing ovulation in a ruminant using the formulation is also provided.

### FIELD OF THE INVENTION

This invention relates to the field of ovulation regulation in ruminants.

### BACKGROUND

Dairy cattle operations require efficient breeding regimens for optimal performance and economic yield. Milk production is dependent on cows in the operation becoming pregnant, giving birth and lactating. After birth a cow can be milked for over two hundred days. After about 150 days post parturition, however, milk production typically falls off rapidly. Decreasing the time period between calving and the following pregnancy in a birth-lactation cycle thus increases milk production the value of a cow to the dairy operation. Additionally, the dairy value of a cow typically decreases rapidly thirty six months after first calving. Increasing the number of pregnancies a cow has during this time maximizes the return on investment in the animal in terms of feed, overhead and other costs.

The goals of high milk yield and fertility may be difficult to achieve simultaneously. A high milk yield results in decreasing the duration of estrus, ovulation without signs of estrus, and anovulatory conditions that are directly related to fertility, making the basic reproductive management tool, estrus detection followed by artificial insemination (AI) more difficult to implement efficiently. Efficiency of reproductive management can be increased by the use hormones that regulate and control estrous cycles and ovulation. The estrus cycle in cows can be manipulated by, for example, gonadotropin releasing hormone (GnRH), lutenizing hormone (LH), prostaglandin F₂ₐ, estrogen, progesterone and synthetic analogs of certain of these hormone.

The, OVSYNCH® treatment regimen is a three injection-regiment for regulating the cow estrus cycle. GnRH is injected in a cow at any stage of her breeding cycle. The GnRH stimulates LH, which stimulates ovulation of old follicles and growth of a new follicle. A second injection with prostaglandin is given seven days later after the GnRH injection, effecting the degeneration of any active corpus luteum (which would prevent the cow from ovulating). Two days later, a second injection of GnRH is administered, causing release an egg, i.e., ovulation, from the follicle that had been stimulated by the first GnRH injection. The optimal time for insemination is 16 hours after the third injection, but cows stay fertile. for 24 hours. The OVSYNCH® treatment regimen allows for synchronization of ovulation among simultaneously treated cows. The OVSYNCH® treatment regimen, however, requires each cow to be treated four times, at a relatively precise schedule, increasing the cost of treatment and making treatment of large numbers of animals difficult or impractical. Efforts to modify the OVSYNCH® treatment regimen have been made difficult by the relatively high cost of GnRH and relatively low bioavailability of intravaginally administered GnRH.

### SUMMARY OF INVENTION

This invention relates to the field of ovulation regulation in ruminants.

In certain emobodiments is provided an intravaginal formulation including a carboxylic acid in an amount of 10-20% w/w; an oleaginous base, having a melting temperature between 30° C. and 38° C.; and GnRH or an analog thereof, in an amount equivalent to between about 10 and 600 μg GnRH.

In certain embodiments, GnRH or the analog thereof is present in an amount equivalent to the amount of GnRH between 140 and 550 μg; or in the amount equivalent to the amount of GnRH between 140 and 500 μg; or in the amount equivalent to the amount of GnRH between 140 and 300 μg; or in the amount equivalent to the amount of GnRH between 140 and 250 μg; or in the amount equivalent to the amount of GnRH between 140 and 200 μg; or in the amount equivalent to the amount of GnRH between 200 and 500 μg; or in the amount equivalent to the amount of GnRH between 200 and 400 μg; or in the amount equivalent to the amount of GnRH between 200 and 300 μg; or in the amount equivalent to the amount of GnRH between 280 and 550 μg; or in the amount equivalent to the amount of GnRH between 280 and 500 μg; or in the amount equivalent to the amount of GnRH between 280 and 400 μg.

In certain embodiments, GnRH or the analog thereof is present in the amount of between 140% and 250% of ED70 of the GnRH or the analog thereof when administered intramuscularly to a female ruminant.

In certain embodiments, the carboxylic acid is citric acid, succinic acid, tartaric acid, glycolic acid, ascorbic acid, lactic acid, aspartic acid, dipotassium edetate, or a combination thereof. The oleaginous base may have OHV below 30, preferably, below 20, more preferably, below 15, e.g., between 5 and 15.

In certain embodiments, the female ruminant is a bovine, a caprine, or an ovine.

In certain aspects is provided a method of making a formulation according to any of the embodiments described above, by admixing the GnRH or the analog thereof with the carboxylic acid; grinding the GnRH or the analog thereof and the carboxylic acid; and dispersing the ground GnRH or the analog thereof and the ground carboxylic acid in the oleaginous base.

In another aspect is provided a method of inducing ovulation in a diestrous ruminant, by intravaginally administering a formulation according to any of the preceding embodiments. In certain aspects, the intravaginal formulation is provided as a part of a multi-component formulation, which also comprises prostaglandin F2α and either an effective dose of prostaglandin and another effective dose of GnRH or an analog thereof. In certain aspects, the multi-component formulation is within an intravaginal device, configured to release an another effective dose of GnRH or analog thereof 6-10 days before the release of the prostaglandin F2α, e.g., 7 days, 8, days, or 9 days. An intravaginal device may also be configured to release a formulation as described herein about two days after the release of the prostaglandin F2α. In certain aspects the other effective dose of GnRH or the analog thereof may be identical to the intravaginal formulation described above.

### BRIEF DESCRIPTION OF THE DRAWINGS

FIG. 1a and 1b illustrate least squares means (+/− standard error) of AUC for animals treated with gonadorelin acetate IVg.

FIGS. 2a and 2b illustrate least squares means (+/− standard error) of AUC for animals treated with gonadorelin hydrochloride IVg.

### DETAILED DESCRIPTION

Definitions

For a better understanding of the invention, the following non-limiting definitions are provided:

The term “GnRH” refers to GnRH forms naturally present in a ruminant. Examples of GnRH include, without limitation, bovine GnRH, caprine GnRH, ovine GnRH and salts thereof. GnRH is a decapeptide having amino acid sequence of SEQ ID NO: 1 (pyro-EHWSYGLRPG-NH₂). Generally, acetates, acetate hydrates and/or hydrochlorides of GnRH are used.

The term“ED70” of as applied to GnRH or an analog of GnRH refers to the dose of GnRH or an analog of GnRH, as applicable, that causes ovulation in 70% of bovine ruminants, when administered to the ruminant in diestrus stage of estrous cycle.

The term “amount of GnRH analog equivalent to” a certain GnRH amount refers to a ratio of GnRH amount to the potency of the analog. For example, 2 ng of a GnRH analog that is 50 times more potent than GnRH is equivalent to 100 ng of GnRH.

Formulations

The formulations disclosed herein are useful for intravaginal administration of GnRH to a ruminant, e.g., bovine and ovine species. The inventors have surprisingly and unexpectedly discovered that formulations disclosed herein provide for a bioavailability of GnRH or GnRH analogs approaching (e.g., about 70-75%) the bioavailability obtained upon intramuscular administration. The efficient intravaginal delivery of GnRH obtained using the formulation disclosed herein provides for increased economy of delivering a GnRH or a GnRH analog intravaginally. Additionally, the formulations disclosed herein allow for the design of a “one-touch” protocol for estrous cycle synchronization and ovulation induction in ruminants, i.e. a protocol requiring only a single administration of hormone.

Formulations providing increased bioavailability of GnRH or a GnRH analog include the GnRH or GnRH analog in combination with a carboxylic acid and an oleaginous base. The GnRH or GnRH analog is typically present in an amount equivalent to a GnRH amount of between 10 and 600 μg. In certain embodiments, the formulation of the instant invention contains GnRH or the analog thereof the range of GnRH or the analog thereof in the amount equivalent to GnRH amount of 140-600 μg. In certain instances, the effective amount of GnRH or GnRH analog in an intravaginal formulation may be based on the amount of GnRH known to be effective when administered intramuscularly, adjusted for the respective GnRH bioavailabilities obtained following administration of the intravaginal formulation and intramuscular administration. In the case of a bovine, for example, certain GnRH products recommend 100-200 μg of GnRH per animal, administered intramuscularly. For an intravaginal GnRH formulation providing a bioavailability that is 75% of the bioavailability obtained upon intramuscular administration of GnRH, the formulation would include the equivalent of about 133-266 μg of GnRH. Such formulation may thus provide, for example, 140 μg per animal administered intravaginally.

The amount of GnRH or GnRH analog present in a formulation preferably produces synchronous ovulation when administered intravaginally to a group of animals. Thus, without being bound by theory, intravaginal formulations may include GnRH or a GnRH analog in an amount of between 140% and 300% of ED70 of the GnRH or GnRH analog, when administered intramuscularly to a female ruminant. For example, GnRH or GnRH analog thereof may be present in the amount equivalent to a GnRH amount of 140%- 275%, 140%-250%, 140% - 200%, 180-250%, or 200%-220% of ED70 of the GnRH or the GnRH analog when administered intramuscularly to the female ruminant.

GnRH or a GnRH analog may be present in the amount equivalent to the amount of GnRH between 140 and 550 μg; or in the amount equivalent to the amount of GnRH between 140 and 500 μg; or in the amount equivalent to the amount of GnRH between 140 and 300 μg; or in the amount equivalent to the amount of GnRH between 140 and 250 μg; or in the amount equivalent to the amount of GnRH between 140 and 200 μg; or in the amount equivalent to the amount of GnRH between 200 and 500 μg; or in the amount equivalent to the amount of GnRH between 200 and 400 μg; or in the amount equivalent to the amount of GnRH between 200 and 300 μg; or in the amount equivalent to the amount of GnRH between 280 and 550 μg; or in the amount equivalent to the amount of GnRH between 280 and 500 μg; or in the amount equivalent to the amount of GnRH between 280 and 400 μg.

Intravaginal GnRH formulations may include, GnRH and/or one or more GnRH salt and/or one or more GnRH analog. GnRH salts include, for example and without limitation, GnRH hydrochloride, GnRH acetate and GnRH acetate tetrahydrate. GnRH analogs include, for example arid without limitation, leuprolide (pyrGlu-His-Trp-Ser-Tyr-D-Leu-Leu-Arg-Pro-NHEt; SEQ ID NO: 2), buserelin (pyrGlu-His-Trp-Ser-Tyr- D-Ser(Tbu) -Leu-Arg-Pro-NHEt; SEQ ID NO: 3), deslorelin (pyrGlu-His-Trp-Ser-Tyr-D-Trp-Leu-Arg-Pro-NHEt; SEQ ID NO: 4) and fertirelin (pyrGlu-His-Trp-Ser-Tyr-Gly-Leu-Arg-Pro-NHet; SEQ ID NO: 5), as well as salts and/or hydrates of these analogs. A preferred GnRH analog is buserelin. A preferred dose of buserelin is an amount equal to 10% of GnRH amount.

GnRH analogs are well known in the art. The ovulation-inducing activities of GnRH analogs have been well documented in publicly available sources, such as, for example, the Pubmed database. Further, suitable models for determination of ovulation-inducing activity of GnRH analogs have been developed. Additional examples of GnRH analogs and their ovulation inducing activity are disclosed in Dutta et al, Biochem Biophys Res. Commun. 1978 81(2): 382-390; Fujino et al, Biochem Biophys Res. Common 1974 57(4): 12484256; Dutta et al, J Med. Chem. 1978 21(10): 1018-1024; Fujino et al, Biochem Biophys Res. Commun 1972 49(3): 863-869. Thus, no more than routine experimentation is needed to determine ovulation-inducing activity of GnRH analog(s) of interest.

Multiple oleaginous bases are suitable for the GnRH intravaginal formulations described herein. Generally, oleaginous bases contain mixtures of mono- di- and triglycderides. The relative amounts of mono-, di- and triglycerides, as well as the lengths of fatty acid tails influence OHV value (content of free hydroxyl groups). In other words, OHV value provides certain information about relative amounts of mono-, di- and triglycerides in the oleaginous base. In certain embodiments, the oleaginous base has OHV value below 30. Thus, in different embodiments, the OHV value is below 25, below 20, below 15, below 10, or below 5. In some embodiments, OHV value of the oleaginous base is between about 5 and about 15.

Examples of oleaginous bases are the WITEPSOL® series suppositories. The OHV value of WITEPSOL® H 15 suppository is between 5 and 15. H series are hard fats with hydroxyl values up to 15. They consist mostly of triglycerides with a proportion of, at most, 15% of diglycerides and not more than 1% of monoglycerides. They are characterized by a very small gap between the melting and solidification temperatures, have only a small tendency to the posthardening phenomenon (maximum 1.5 ° C.).

WITEPSOL® W series products are hard fats with hydroxyl values (OHV) of 20-50. They consist of a mixture of triglycerides (65-80%), diglycerides (10-35%) and monoglycerides (1-5%). As a result of their composition, these WITEPSOL grades have a larger gap between melting and solidification points, they are less sensitive to shock cooling (more elastic), solidify more slowly and can be readily processed both with automatic machines and with small scale equipment. The partial glyceride content also slows down the sedimentation of solids and promotes the absorption of less readily absorbable active compounds.

In certain aspects, the oleaginous base has OHV value below 30. Thus, the OHV value may be below 25, below 20, below 15, below 10, or below 5. In some aspects, OHV value of the oleaginous base is between about 5 and about 15.

The oleaginous base may also have a melting temperature below the body temperature of the ruminant. %he melting temperature of the oleaginous base may also be in the range of, for example, between 30° C. and 40° C., e.g., 30.5-32.5° C., 32.5-34.5 ° C., 34.5-36.5 ° C., 36.5-38.5 ° C., and 38.540° C.

Different carboxylic acids are suitable for the GnRH intravaginal formulations described herein. Without limitations, exemplary carboxylic acids include citric acid, succinic acid, tartaric acid, glycolic acid, ascorbic acid, lactic acid, aspartic acid, dipotassium edetate, or a combination thereof. Generally, GnRH intravaginal formulations contain about 8-20% w/w of the carboxylic acid (e.g., 9%, 10%, 11%, 12%, 13%, 14%, 15%, 16%, 17%, 18%, 19%).

, GnRH intravaginal formulations may be prepared by dispersing a solid form of GnRH or a GnRH analog and a solid form of a carboxylic acid in an oleaginous base. The GnRH or GnRH analog and/or the carboxylic acid may be processed (for example, ground) to reduce particle size, prior to being dispersed in the oleaginous base.

, A protocol for synchronizing of ovulation in bovine may be effected, without limitation, by administering one or more bovines GnRH or a GnRH analog thereof (referred to as “GnRH-1”), administering the bovines Prostaglandin F 2a within 5-9 days thereafter, and subsequently administering the bovines a second dose of GnRH or GnRH analog (referred to as “GnRH-2”) about 30-72 hours after prostaglandin administration. Without being bound by theory, GnRH-1 administration induces ovulation or luteinization of the dominant follicle that standardizes follicular wave emergence and follicular growth patterns in the majority of animals treated. GnRH-2 administration (which induces ovulation of the dominant follicle). The GnRH intravaginal formulations described are suitable for either or both of GnRH-1 and/or GnRH-2 administration.

GnRH intravaginal formulations described herein may be used as a part of a multi-component composition. Such a multi-component may comprise a synchronization component, a prostaglandin component, and an ovulation-inducing component. The GnRH intravaginal formulations described herein are thus suitable for use as a synchronization component, an ovulation-inducing component, or both a synchronization component and an ovulation-inducing component. A multi-component composition may loaded within an intravaginal device configured to deliver the treatments, as desired by a practitioner. An intravaginal delivery device may be configured, for example, to first a synchronization component, a prostaglandin component (6-10 days after the synchronization component has been delivered), and an ovulation-inducing component (30--72 hours after the prostaglandin component has been delivered).

In certain aspects a GnRH intravaginal formulation is used as both a synchronization component and an ovulation-inducing component of the multi-component composition. A prostaglandin component is delivered about 7 days after the synchronization component, and an ovulation-inducing component is delivered 30-72 hours after the prostaglandin component.

A multi-component composition may be administered advantageously from within a device configured for timed delivery of different components. Such a device may contain, for example, one or more reservoirs and a programmable processing unit. Respective reservoirs may contain a synchronization component, an ovulation-inducing component, and a prostaglandin component. At predetermined times, the programmable processing unit effects release of the appropriate component from a reservoir of the device into the vagina of the ruminant. A programmable processing unit effect such a release via a rupture of a wall of a reservoir and extrusion, e.g., through electrical, mechanical, or osmotic force, of the contents of the reservoir out of the device.

All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

The invention is further explained in non-limiting Examples.

### EXAMPLES

### Example 1

### Bioavailability of Intravaginal Gonadorelin Acetate

Materials and Methods

Animals and treatments

Animals were adult, lactating, holstein cattle. They were between first and third parity, a minimum of 50 days post-partum and were not pregnant. These animals were group housed in a modern free stall dairy facility with sand bedding and they were milked twice daily. Prior to study initiation animals were allocated to one of four treatment groups according to a randomized complete block design with one-way treatment structure: They were blocked according to bodyweight which ranged from 500-834 kg. Treatment 1 (T01) “positive control” (n=4) received 220 μg gonadorelin acetate (200 μg-equivalent (“eq”) gonadorelin) in PBS via Intramuscular (IM) administration; Treatment 2 (T02) received 4406 μg of gonadorelin acetate (4000 μg-eq gonadorelin) mixed with 40 mg citric acid intravaginally (IVg) as a powder; Treatment 3 (T03) received 4406 μg gonadorelin acetate with 40 mg citric acid compressed into a pellet IVg; Treatment 4 (T04) received 4406 μg gonadorelin acetate with 40 mg citric acid dispersed in a waxy matrix (WITEPSOL® H-15) IVg. All treatments were administered under a Zoetis IACUC approved animal use protocol.

Formulation

Treatment 1 consisted of 1.37 mg of gonadorelin acetate that was dissolved in 25 mL of phosphate buffered saline. The resultant 55 μg/mL solution is equivalent to 50 μg/mL of gonadorelin. This solution was sterilized by filtering through a 0.22 μm filter. Four mL of this solution was administered to achieve a total dose of 200 μg of gonadorelin. Treatment 2 consisted of 4406 μg of gonadorelin acetate that was mixed with 400 mg of citric acid and then ground to achieve a smaller, more uniform particle size. This was administered as a loose powder which is equivalent to 4000 μg of gonadorelin. Treatment 3 consisted of 4406 μg of gonadorelin acetate that was mixed with 320 mg of citric acid, 960 mg of lactose, 960 mg of microcrystalline, 160 mg of crospovidone and 6 mg of magnesium stearate. This mixture was then ground to achieve a smaller, more uniform particle size and 305 mg of mixture was compressed into pellet form to make each dose. Each pellet contained 4406 μg of gonadorelin acetate which is equivalent to 4000 μg of gonadorelin. Treatment 4 consisted of suppositories created by mixing 4406 μg of gonadorelin acetate with 40 mg of citric acid. The mixture was ground to achieve a smaller more uniform particle size and then it was dispensed in 260 mg of WITEPSOL° H15 that was melted at 60° C. in a 2 mL plastic tube. This suspension was vortexed to ensure homogeneity. It was then allowed to cool to form a solid suppository containing a dose equivalent to 4000 μg gonadorelin.

Experimental Design

Animals were examined prior to study initiation to ensure that they were clinically healthy, with no lameness present and had a normal reproductive tract. Milk was screened to ensure that somatic cell counts were below 750,000 cells/mL and mammary quarters were mastitis free. Bodyweights were collected to randomize animals to treatment. Basal custom blended feed rations representative of the industry for lactation were fed and a description of the diet was documented in the study data. Water was provided ad libitum. Estrus was not synchronized prior to the initiation of this study. All treatments were administered on the same day. Treatment 1 was administered as an IM bolus into the shoulder region of each animal. Treatment 2 was administered as a bolus into the vagina with a modified equine intrauterine pipette. Treatments 3 and 4 were administered as a bolus into the vagina with a modified goat oral balling gun. Blood samples were collected prior to dosing and at 0.33, 0.75, 1, 1.5, 2, 4, 7, 12, and 24 hours post-dose. Plasma was isolated from these samples and processed for GnRH analysis.

Plasma GnRH

After collecting blood into prechilled K2 EDTA tubes containing 0.6 mL of 3 mM bacitracin to act as a protease inhibitor, samples were mixed by inverting the tubes at least 5 times. The samples were then placed on ice and centrifuged at 4° C. within 15 minutes of collection. 0.6 mL of plasma was then added to 2.0 mL of methanol and the sample was vortexed for 60 seconds. Samples were frozen at −20° C. and shipped to Endolytics, LLC for analysis by RIA as described previously (Nett, T. M., A. M. Akbar, W. R. White, M. T. Hedlund and G. D. Niswender. 1973. Radioimmunoassay for gonadotropin-releasing hormone (GnRH) in serum. J. Clin. Endocrinol. Metab. 36:880-885).

Results

Group Mean Plasma Gonadorelin Concentrations

As demonstrated in Table 1, animals receiving 4400 μg IVg doses (T02, T03, T04; 20x IM dose) achieved higher group mean plasma gonadorelin concentrations than animals receiving 0.22 mg IM doses T01). Of those treatments that received IVg doses (T02, T03, T04), T04 experienced the highest mean plasma concentration. Mean gonadorelin concentrations for the T02 and T04 IVg administrations were higher than the IM T01 administration for 12 and 7 hours post-dose respectively. Mean gonadorelin concentrations were higher for the T03 IVg administration than those of T01 from approximately 45 minutes post-dose until 7 hours post-dose. Plasma gonadorelin concentrations were highest in T04 followed by T02, then T03 and T01 respectively (n=4 for all treatments). Treatment 2, T03, and T04 received a dose that was 20× greater than the dose administered to T01. By 24 hours post-dose administration plasma gonadorelin levels for all treatments were below the limit of quantitation.

TABLE 1
Group Mean Plasma Gonadorelin Concentration

| | gonadore in (pg/mL) | | | | | | | | | |
|---|---|---|---|---|---|---|---|---|---|---|
| | | 0.333 | 0.75 | | 1.5 | | | | 12 | 24 |
| Treatments | 0 h | h | h | 1 h | h | 2 h | 4 h | 7 h | h | h |
| T01 | 19 | 124 | 83 | 49 | 22 | 16 | 0.80 | 0 | 0 | 0 |
| 220 μg | | | | | | | | | | |
| IM | | | | | | | | | | |
| T02 | BLQ | 1050 | 694 | 627 | 413 | 263 | 200 | 66 | 26 | 0 |
| 4400 μg | | | | | | | | | | |
| IVg | | | | | | | | | | |
| T03 | BLQ | 17 | 86 | 333 | 440 | 293 | 112 | 16 | 0 | 0 |
| 4400 μg | | | | | | | | | | |
| IVG | | | | | | | | | | |
| T04 | BLQ | 1510 | 1260 | 836 | 453 | 192 | 33.4 | 1.48 | 0 | 0 |
| 4400 μg | | | | | | | | | | |
| IVG | | | | | | | | | | |

T01: 220 μg Gonadorelin acetate in PBS (200 μg-eq gonadorelin)
T02: 4400 μg Gonadorelin acetate + 40 mg citric acid (powder) (4000 μg-eq gonadorelin)
T03: 4400 μg Gonadorelin acetate + 40 mg citric acid (pellet) (4000 μg-eq gonadorelin)
T04: 4400 μg Gonadorelin acetate + 40 mg citric acid in wax (suppository) (4000 μg-eq gonadorelin)
BLQ—BELOW QUANTITATION LIMIT

Group Mean Pharmacokinetic Parameters

As indicated in table 2, animals receiving 4400 μg IVg doses (T02, T03, T04) achieved higher mean Cmax and mean AUC (exposure) than animals receiving 220 μg IM doses T01) as was expected due to 20× higher doses.

The highest mean Cmax was achieved by T04 (IVg), which was approximately 15× greater than that of the T01 IM dose and represented a relative Cmax of 75% of IM dose (after dose normalization) The mean Cmax values for T02 and T03 were approximately 9× and 5× greater than T01, respectively. Cmax as a ratio of group mean Cmax to IM Cmax was greatest in T04, followed by T02, T03, and then T01 respectively (n=4 for all treatments). Bioavailability (F) and AUC as a ratio of group mean AUC to IM AUC was greatest in T02, followed by T04, T03 and then T01 respectively. Treatment 2, T03, and T04 received doses that were 20× greater than the dose administered to T01.

The IVg bioavailability (relative to IM dose) was 92%, 42%, and 72% for T02, T03, and T04 respectively. The IVg bioavailability was greatest in T02, although Cmax and Tmax may be more pertinent when determining bioactivity of gonadorelin or its analogues.

IM administration of T01 resulted in a Tmax (20 min) that was earlier than any of the IVg treatments. Of the IVg administrations, T04 exhibited the earliest Tmax at approximately 26 minutes with T02 and T03 reaching their respective Tmax at approximately 37 and 90 minutes

TABLE 2
Mean Plasma PK for Gonadorelin

| | | Cmax | | | | | AUC | | | | |
|---|---|---|---|---|---|---|---|---|---|---|---|
| Treatment | | (pg/mL) | | Cmax/ | Tmax (h) | | (pg*h/mL) | | AUC/ | F | |
| Group | Dose (μg) | Mean | S.D. | Cmax, im | Mean | S.D. | Mean | S.D. | AUCim | Mean | S.D. |
| T01 | 220 IM | 124 | 34 | — | 0.33 | 0.00 | 129 | 20 | — | — | — |
| T02 | 4400 IVg | 1070 | 746 | 8.6 | 0.62 | 0.59 | 2380 | 754 | 19.2 | 0.92 | 0.29 |
| T03 | 4400 IVg | 568 | 463 | 4.6 | 1.50 | 0.41 | 1090 | 691 | 8.8 | 0.42 | 0.27 |
| T04 | 4400 IVg | 1850 | 1150 | 14.9 | 0.44 | 0.21 | 1850 | 1130 | 14.9 | 0.72 | 0.44 |

T01: 220 μg Gonadorelin acetate in PBS (200 μg-eq gonadorelin)
T02: 4400 μg Gonadorelin acetate + 40 mg citric acid (powder) (4000 μg-eq gonadorelin)
T03: 4400 μg Gonadorelin acetate + 40 mg citric acid (pellet) (4000 μg-eq gonadorelin)
T04: 4400 μg Gonadorelin acetate + 40 mg citric acid in wax (suppository) (4000 μg-eq gonadorelin)
F = IVg bioavailability relative to IM dose

### Example 2

### Dose Linearity of Intravaginal Administration of Gonadorelin Acetate

Materials and Methods

Animals and treatments

Animals were adult, lactating, holstein cattle. They were between first and third parity, a minimum of 50 days post-partum and were not pregnant. These animals were group housed in a modern free stall dairy facility with sand bedding and they were milked twice daily. Prior to study initiation animals were allocated to one of four treatments according to a randomized complete block design with one-way treatment structure: They were blocked according to bodyweight which ranged from 518-782 kg. Treatment 1 “positive control” (n=4) received 220 μg gonadorelin acetate (200 μg-eq gonadorelin) in PBS via Intramuscular (IM) administration; Treatment 2 received 220 μg gonadorelin acetate (200 μg-eq gonadorelin) with 40 mg citric acid dispersed in a waxy matrix (WITEPSOL° H-15) IVg ; Treatment 3 received 440 μg gonadorelin acetate (400 μg-eq gonadorelin) with 40 mg citric acid dispersed in a waxy matrix (WITEPSOL° H-15) IVg; Treatment 4 received 880 μg gonadorelin acetate (800 μg-eq gonadorelin) with 40 mg citric acid dispersed in a waxy matrix (WITEPSOL° H-15) IVg. All treatments were administered under a Zoetis IACUC approved animal use protocol.

Formulation

Treatment 1 consisted of 1.37 mg of gonadorelin acetate that was dissolved in 25 mL of phosphate buffered saline. The resultant 55 μg/mL solution is equivalent to 50 μg/mL of gonadorelin. This solution was sterilized by filtering through a 0.22 μm filter. Four mL of this solution was administered to achieve a total dose of 200 μg of gonadorelin. Treatments 2 through 4 consisted of suppositories created by mixing 220 μg, 440 μg, or 880 μg of gonadorelin acetate respectively, with 40 mg of citric acid. The mixture was ground to achieve a smaller more uniform particle size and then it was dispensed in 260 mg of Witepsol H15 that was melted at 60° C. in a 2 mL plastic tube. This suspension was vortexed to ensure homogeneity. It was then allowed to cool to form a solid suppository containing a dose equivalent to 200 μg, 400 μg, or 800 μg of gonadorelin respectively.

Experimental Design

Animals were examined prior to study initiation to ensure that they were healthy, with no lameness present and had a normal reproductive tract. Milk was screened to ensure that somatic cell counts were below 750,000 cells/mL and mammary quarters were mastitis free. Bodyweights were collected to randomize animals to treatment. Basal custom blended feed rations representative of the industry for lactation were fed and a description of the diet was documented in the study data. Water was provided ad libitum. Estrus was not synchronized prior to the initiation of this study. All treatments were administered on the same day. Treatment 1 was administered as an IM bolus into the shoulder region of each animal. Treatments 2, 3 and 4 were administered as a bolus into the vagina with a modified goat oral balling gun. Blood samples were collected prior to dosing and at 0.167, 0.33, 0.75, 1, 1.5, 2, 4, and 7 hours post-dose. Plasma was isolated and processed for GnRH analysis.

Plasma GnRH

After collecting blood into prechilled K2 EDTA tubes containing 0.6 mL of 3 mM bacitracin to act as a protease inhibitor, samples were mixed by inverting the tubes at least 5 times. The samples were then placed on ice and centrifuged at 4° C. within 15 minutes of collection. 0.6 mL of plasma was then added to 2.0 mL of methanol and the sample was vortexed for 60 seconds. Samples were frozen at −20° C. and shipped to Endolytics, LLC for analysis by RIA as described previously (Nett, T. M., A. M. Akbar, W. R. White, M. T. Hedlund and G.D. Niswender. 1973. Radioimmunoassay for gonadotropin-releasing hormone (GnRH) in serum. J. Clin. Endocrinol. Metab. 36:880-885).

Results

Back Transformed Group Mean Plasma Gonadorelin Concentrations

As indicated in Table 3, animals receiving IVg doses at 440 μg and 880 μg (T03 and T04 respectively) achieved higher peak group mean plasma gonadorelin concentrations than animals receiving 220 μg IM doses T01). The peak plasma concentrations demonstrated by the IVg treatments (T02, T03 and T04) were achieved later than the highest plasma concentrations achieved by administration of T01 (0.75 hr vs 0.333 hr respectively). While animals in T01 and T02 received equivalent doses of gonadorelin acetate, plasma concentrations for the IVg route of administration T02) never reached the same level as the plasma levels exhibited after the IM administration T01). Plasma gonadorelin concentrations were highest in T04 followed by T03, then T01 and T02 respectively (n=4 for all treatments). By 4 hours post-dose, plasma gonadorelin levels for all treatments were below the limit of quantitation.

TABLE 3
Back Transformed Mean Plasma Gonadorelin Concentration

| | gonadorelin (pg/mL) | | | | | | | | |
|---|---|---|---|---|---|---|---|---|---|
| | | 0.167 | 0.333 | 0.75 | | 1.5 | | | |
| Treatments | 0 h | h | h | h | 1 h | h | 2 h | 4 h | 7 h |
| T01 | BLQ | 63 | 105.4 | 57.3 | 47.5 | 27.6 | 13.5 | BLQ | BLQ |
| 220 μg | | | | | | | | | |
| IM | | | | | | | | | |
| T02 | BLQ | 8.2 | 26.8 | 45.0 | 27.6 | 16.7 | 7.7 | BLQ | BLQ |
| 220 μg | | | | | | | | | |
| IVg | | | | | | | | | |
| T03 | BLQ | 19.7 | 65.6 | 114.8 | 51.5 | 34.8 | 12.6 | BLQ | BLQ |
| 440 μg | | | | | | | | | |
| IVG | | | | | | | | | |
| T04 | BLQ | 25.1 | 92.7 | 166.0 | 99.1 | 64.9 | 31.3 | BLQ | BLQ |
| 880 μg | | | | | | | | | |
| IVG | | | | | | | | | |

T01: 220 μg Gonadorelin acetate in PBS (200 μg-eq gonadorelin)
T02: 220 μg Gonadorelin acetate + 40 mg citric acid in wax (suppository) (200 μg-eq gonadorelin)
T03: 440 μg Gonadorelin acetate + 40 mg citric acid in wax (suppository) (400 μg-eq gonadorelin)
T04: 880 μg Gonadorelin acetate + 40 mg citric acid in wax (suppository) (800 μg-eq gonadorelin)
BLQ—Below Quantitation limit (<1.9 pg/mL)

Group Mean Pharmacokinetic Parameters

As indicated in Table 4, animals receiving IVg doses of 440 μg and 880 μg (T03, T04) achieved higher mean Cmax and AUC levels than animals receiving 220 μg doses either IMT01) or IVg T02). Among those animals receiving IVg administrations, the highest mean Cmax and AUC was achieved by T04, with a Cmax that was approximately 1.8 fold greater than that of the T01 IM administration and an AUC that was approximately 2.3 fold greater. Treatment 3 followed with a mean Cmax and AUC that were approximately equal to that of T01. IVg administrations T02) that were equivalent to the IM dose of 220 μg T01) resulted in a Cmax and AUC that were approximately 43% and 49% of the IM dose respectively. As expected, IM administration of gonadorelin T01) result in a Tmax that was earlier than that of any other treatment, at approximately 20 minutes post-dose. Of the IVg administrations, T04 exhibited the second earliest Tmax at approximately 36 minutes with T02 and then T03 reaching their respective Tmax at approximately 45 minutes.

TABLE 4
Least Squares Means Plasma PK for Gonadorelin

| | | Cmax (pg/mL) | | Tmax (h) | | AUC (pg*h/mL) | |
|---|---|---|---|---|---|---|---|
| Treatment | | Least Squares Means | | Least Squares Means | | Least Squares Means | |
| Group | Dose (μg) | Mean | S.E. | Mean | S.E. | Mean | S.E. |
| T01 | 220 μg IM | 105 | 35.8 | 0.330 | 0.0757 | 112 | 44.4 |
| T02 | 220 μg IVg | 45 | 15.2 | 0.750 | 0.0757 | 55 | 21.8 |
| T03 | 440 μg IVg | 115 | 46.4 | 0.750 | 0.0874 | 116 | 55.6 |
| T04 | 880 μg IVg | 191 | 65.0 | 0.605 | 0.0757 | 260 | 102.9 |

T01: 220 μg Gonadorelin acetate in PBS (200 μg-eq gonadorelin)
T02: 220 μg Gonadorelin acetate + 40 mg citric acid in wax (suppository) (200 μg-eq gonadorelin)
T03: 440 μg Gonadorelin acetate + 40 mg citric acid in wax (suppository) (400 μg-eq gonadorelin)
T04: 880 μg Gonadorelin acetate + 40 mg citric acid in wax (suppository) (800 μg-eq gonadorelin)

Dose Proportionality

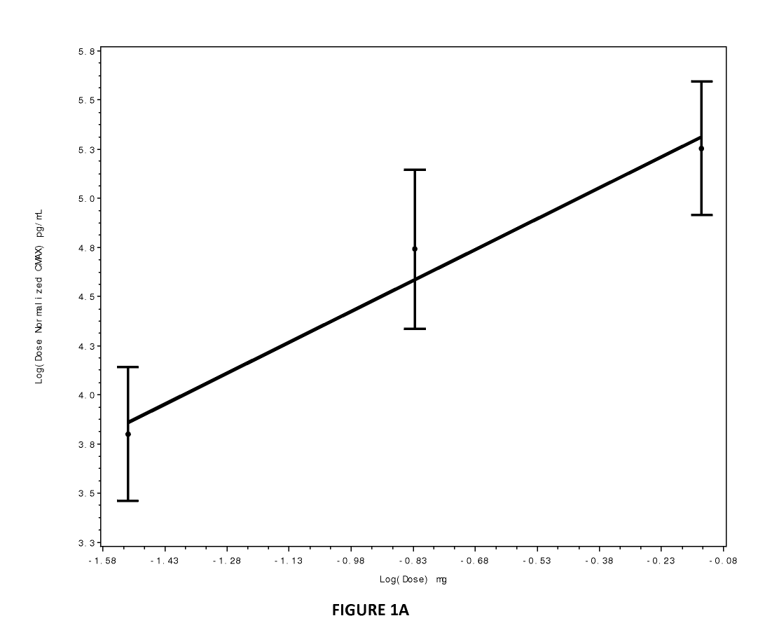

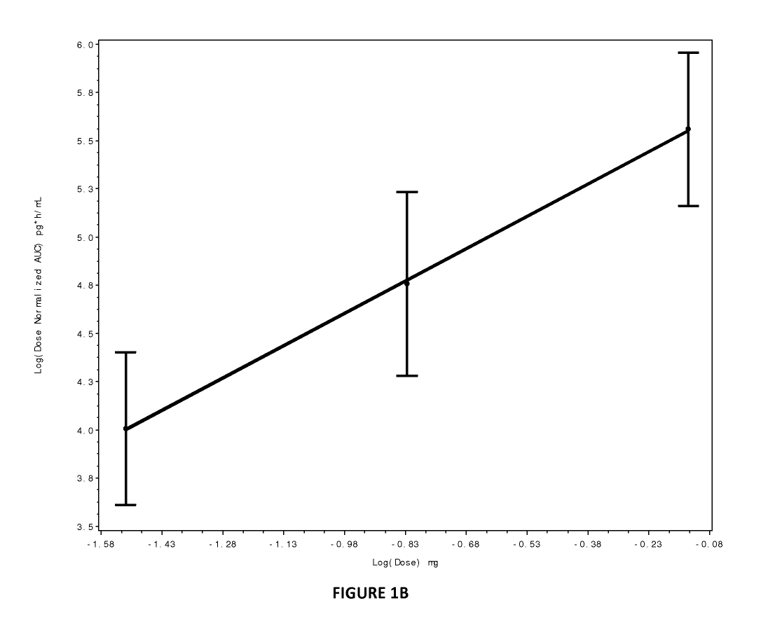

As indicated by FIGS. 1A and 1B, a formulation of gonadorelin acetate, in conjunction with citric acid and Witepsol H15, will provide a dose proportional Cmax and AUC when administered intravaginally. This proportionality allows for an estimate of the IVg dose required to achieve plasma concentrations of gonadorelin similar to those observed after IM administration of gonadorelin acetate. Tests for the lack of fit yielded p values of 0.6912 (for Cmax) and 0.9686 (for AUC), both non-significant at 0.1 level.

### Example 3

### Dose Linearity of Intravaginal Administration of Gonadorelin Hydrochloride

Materials and Methods

Animals and treatments

Animals were adult, lactating, holstein cattle. They were between first and third parity, a minimum of 50 days post-partum and were not pregnant. These animals were group housed in a modern free stall dairy facility with sand bedding and they were milked twice daily. Prior to study initiation animals were allocated to one of four treatments according to a randomized complete block design with one-way treatment structure: They were blocked according to bodyweight which ranged from 539-781 kg. Treatment 1 T01) “positive control” (n=4) received 212.3 μg gonadorelin HCl (200 μg-eq gonadorelin) in PBS via Intramuscular (IM) administration; Treatment 2 T02) received 212.3 μg gonadorelin HCl (200 μg-eq gonadorelin) with 40 mg citric acid dispersed in a waxy matrix (WITEPSOL° H-15) IVg ; Treatment 3 (T03) received 424.6 μg gonadorelin HCl (400 μg-eq gonadorelin) with 40 mg citric acid dispersed in a waxy matrix (WITEPSOL° H-15) IVg; Treatment 4 (T04) received 849.2 μg gonadorelin HCl (800 μg-eq gonadorelin) with 40 mg citric acid dispersed in a waxy matrix (WITEPSOL° H-15) IVg. All treatments were conducted under a Zoetis IACUC approved animal use protocol.

Formulation

T01 consisted of 1.37 mg of gonadorelin HCl that was dissolved in 25 mL of phosphate buffered saline. The resultant 53.1 μg/mL solution is equivalent to 50 μg/mL of gonadorelin. This solution was sterilized by filtering through a 0.22 μm filter. Four mL of this solution was administered to achieve a total dose of 200 μg of gonadorelin. T02, T03 and T04 consisted of suppositories created by mixing 212.3 μg, 424.6 μg, or 849.2 μg of gonadorelin HCl respectively, with 40 mg of citric acid. The mixture was ground to achieve a smaller more uniform particle size and then it was dispensed in 260 mg of Witepsol H15 that was melted at 60° C. in a 2 mL plastic tube. This suspension was vortexed to ensure homogeneity. It was then allowed to cool to form a solid suppository containing a dose equivalent to 200 μg, 400 μg, or 800 μg of gonadorelin respectively.

Experimental Design

Animals were examined prior to study initiation to ensure that they were healthy, with no lameness present and had normal reproductive tracts. Milk was screened to ensure that somatic cell counts were below 750,000 cells/mL and mammary quarters were mastitis free. Bodyweights were collected to randomize animals to treatment. Basal custom blended feed rations representative of the industry for lactation were fed and a description of the diet was documented in the study data. Water was provided ad libitum. Estrus was not synchronized prior to the initiation of this study. All treatments were administered on the same day. T01 was administered as an IM bolus into the shoulder region of each animal. T02, T03 and T04 were administered as a bolus into the vagina with a modified goat oral balling gun. Blood samples were collected prior to dosing and at 0.167, 0.33, 0.75, 1, 1.5, 2, 4, and 7 hours post-dose. Plasma was isolated and processed for GnRH analysis.

Plasma GnRH

After collecting blood into prechilled K2 EDTA tubes containing 0.6 mL of 3 mM bacitracin to act as a protease inhibitor, samples were mixed by inverting the tubes at least 5 times. The samples were then placed on ice and centrifuged at 4° C. within 15 minutes of collection. 0.6 mL of plasma was then added to 2.0 mL of methanol and the sample was vortexed for 60 seconds. Samples were frozen at −20° C. and shipped to Endolytics, LLC for analysis by RIA as described previously (Nett, T. M., A. M. Akbar, W. R. White, M. T. Hedlund and G. D. Niswender. 1973. Radioimmunoassay for gonadotropin-releasing hormone (GnRH) in serum. J. Clin. Endocrinol. Metab. 36:880-885).

Results

Back Transformed Group Mean Plasma Gonadorelin Concentrations

As indicated in Table 5, animals receiving an IVg dose of 849.2 μg (T04) achieved a peak group mean plasma gonadorelin concentration that was approximately 79% that of animals receiving a 212.3 μg IM dose (T01). The highest plasma concentrations demonstrated by T02 and T03 were approximately 20% and 46% of T01 respectively. While animals in T01 and T02 received equivalent doses of gonadorelin HCl, concentrations for those animals in which the dose was administered IVg (T02) never reached the concentrations of those animals in which the dose was administered IM (T01). All treatments that received IVg doses of gonadorelin HCl (T02, T03, and T04) also demonstrated peak plasma concentrations of gonadorelin later than those animals that received gonadorelin HCl via the IM route of administration (T01). By 4 hours post-dose, plasma gonadorelin levels for all treatments were below the limit of quantitation.

TABLE 5
Back Transformed Mean Plasma Gonadorelin Concentration

| | gonadorelin (pg/mL) | | | | | | | | |
|---|---|---|---|---|---|---|---|---|---|
| | | 0.167 | 0.333 | 0.75 | | 1.5 | | | |
| Treatments | 0 h | h | h | h | 1 h | h | 2 h | 4 h | 7 h |
| T01 | BLQ | 141.2 | 113.7 | 67.0 | 34.7 | 12.8 | 3.8 | BLQ | BLQ |
| 212.3 μg | | | | | | | | | |
| IM | | | | | | | | | |
| T02 | BLQ | 12.7 | 28.1 | 25. | 15.8 | 8.0 | 4.0 | BLQ | BLQ |
| 212.3 μg | | | | | | | | | |
| IVg | | | | | | | | | |
| T03 | BLQ | 17.5 | 28.3 | 64.4 | 35.5 | 25.4 | 10.8 | BLQ | BLQ |
| 424.6 μg | | | | | | | | | |
| IVg | | | | | | | | | |
| T04 | BLQ | 19.2 | 52.2 | 112.2 | 75.9 | 54.3 | 23.0 | BLQ | BLQ |
| 849.2 μg | | | | | | | | | |
| IVg | | | | | | | | | |

T01: 212.3 μg Gonadorelin HCl in PBS (200 μg-eq gonadorelin)
T02: 212.3 μg Gonadorelin HCl + 40 mg citric acid in wax (suppository) (200 μg-eq gonadorelin)
T03: 424.6 μg Gonadorelin HCl + 40 mg citric acid in wax (suppository) (400 μg-eq gonadorelin)
T04: 849.2 μg Gonadorelin HCl + 40 mg citric acid in wax (suppository) (800 μg-eq gonadorelin)
BLQ—Below quantification limit ( <1.9 pg/mL)

Group Mean Pharmacokinetic Parameters

As indicated in Table 6, animals receiving an IVg dose of 849.2 μg (T04) achieved a higher mean AUC level than animals receiving 212.3 μg doses either IM(T01) or IVg (T02). Among those animals receiving IVg administrations the highest mean Cmax and AUC was achieved by T04, with a Cmax that was approximately 85% that of the T01 IM administration and an AUC that was approximately 1.5 fold greater. T03 followed with a mean Cmax and AUC that were roughly half that of T01. The IVg administration (T02) that was equivalent to the IM dose of 212.3 μg (T01) resulted in a Cmax and AUC that were approximately 21% and 32% of the IM dose respectively. As expected, IM administration of gonadorelin (T01) result in a Tmax that was earlier than that of any other treatment, at approximately 13 minutes post-dose. Of the IVg administrations, T02 exhibited the second earliest Tmax at approximately 32 minutes with T03 and T04 reaching their respective Tmax at approximately 39 minutes.

TABLE 6
Least Squares Means Plasma PK for Gonadorelin

| | | Cmax (pg/mL) | | Tmax (h) | | AUC (pg*h/mL) | |
|---|---|---|---|---|---|---|---|
| Treatment | | Least Squares Means | | Least Squares Means | | Least Squares Means | |
| Group | Dose (μg) | Mean | S.E. | Mean | S.E. | Mean | S.E. |
| T01 | 212.3 μg IM | 144 | 68.5 | 0.210 | 0.0979 | 106 | 56.1 |
| T02 | 212.3 μg IVg | 30 | 14.5 | 0.540 | 0.0979 | 34 | 18.2 |
| T03 | 424.6 μg IVg | 71 | 33.5 | 0.645 | 0.0979 | 77 | 40.8 |
| T04 | 849.2 μg IVg | 123 | 58.3 | 0.645 | 0.0979 | 157 | 83.3 |

T01: 212.3 μg Gonadorelin HCl in PBS (200 μg-eq gonadorelin)
T02: 212.3 μg Gonadorelin HCl + 40 mg citric acid in wax (suppository) (200 μg-eq gonadorelin)
T03: 424.6 μg Gonadorelin HCl + 40 mg citric acid in wax (suppository) (400 μg-eq gonadorelin)
T04: 849.2 μg Gonadorelin HCl + 40 mg citric acid in wax (suppository) (800 μg-eq gonadorelin)

Dose Proportionality

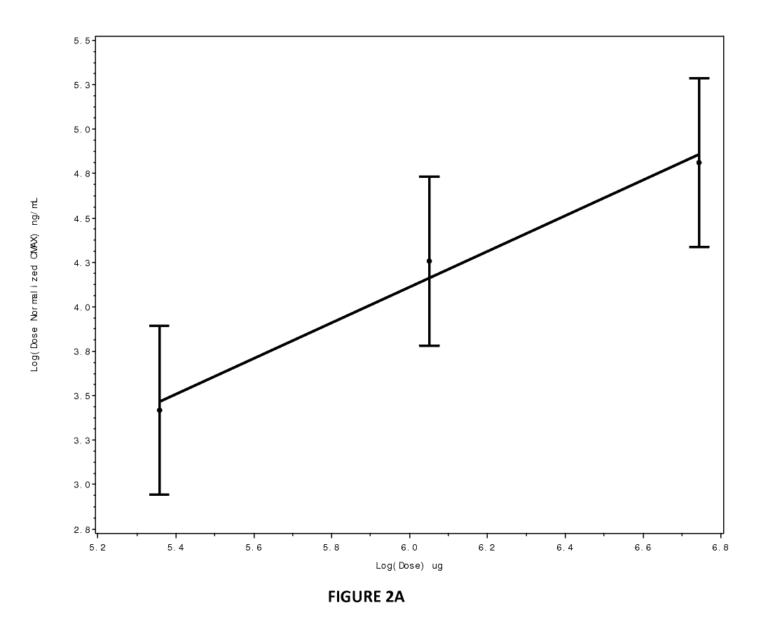

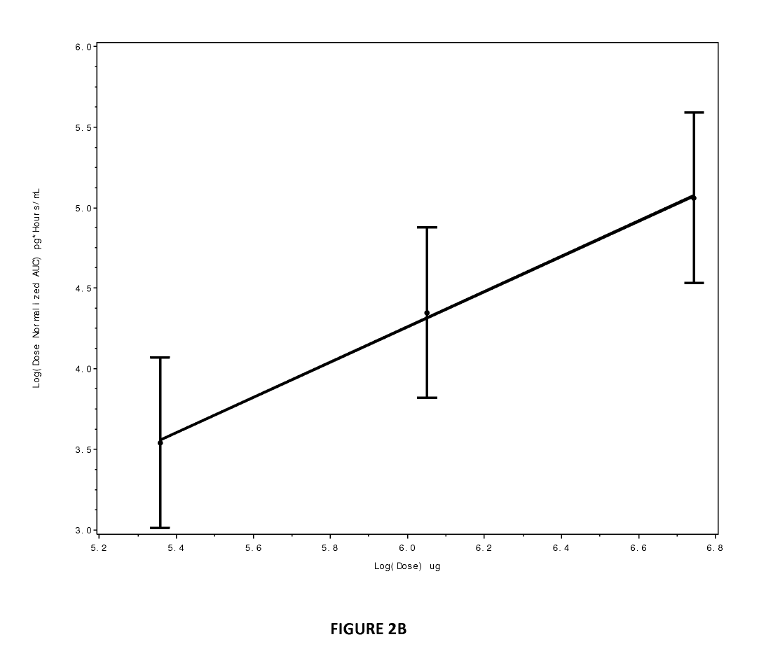

As indicated by FIGS. 2a and 2b, a formulation of gonadorelin HCl, in conjunction with citric acid and Witepsol H15, will provide a dose proportional Cmax and AUC when administered intravaginally. This proportionality allows for an estimate of the IVg dose required to achieve plasma concentrations of gonadorelin similar to those observed after IM administration of gonadorelin HCl. Tests for the lack of fit yielded p values of 0.8379 (for Cmax) and 0.9616 (for AUC), both non-significant at 0.1 level.

1. An intravaginal formulation comprising:
a) 10-20% (w/w) of carboxylic acid;
b) an oleaginous base having a Tm between about 30° C. and 38° C.; and
c) a GnRH or GnRH analog in an amount equivalent to a GnRH amount of between 10 μg and 600 μg.

2. The intravaginal formulation of claim 1, wherein said GnRH or GnRH analog is present in an amount equivalent to a GnRH amount of:
a) between 140 and 550 μg; or
b) between 140 and 500 μg; or
c) between 140 and 300 μg; or
d) between 140 and 200 μg; or
e) between 200 and 500 μg; or
f) between 200 and 400 μg; or
g) between 200 and 300 μg; or
h) between 280 and 550 μg; or
i) between 280 and 500 μg.

3-10. (canceled)

11. The intravaginal formulation of claim 1, wherein said GnRH or the analog thereof is present in the amount of between 140% and 250% of ED70 of said GnRH or the analog thereof when administered intramuscularly to a female ruminant.

12. The intravaginal formulation of claim 11, wherein the female ruminant is a bovine, an ovine or a caprine.

13. The intravaginal formulation of claim 12, wherein the female ruminant is a bovine.

14. The intravaginal formulation of claim 1, wherein said GnRH or the analog thereof is present at a dose of ED70 or greater when administered intravaginally.

15. The intravaginal formulation of claim 1, wherein the carboxylic acid is selected from the group consisting of citric acid, succinic acid, tartaric acid, glycolic acid, ascorbic acid, lactic acid, aspartic acid, dipotassium edetate, or a combination thereof.

16. The intravaginal formulation of claim 15, wherein said carboxylic acid is citric acid.

17. The intravaginal formulation of claim 1, wherein said carboxylic acid is present in the amount of 12-15% w/w of said formulation.

18. The intravavinal formulation of claim 1, wherein said oleaginous base has OHV below 30.

19. The intravaginal formulation of claim 1, wherein said oleaginous formulation has OHV between 1 and 20.

20. The intravaginal formulation of claim 1, wherein said oleaginous formulation has OHV between 5 and 15.

21. A method of inducing ovulation in a female ruminant, comprising administering said female ruminant the intravaginal formulation of claim 1.

22. The method of claim 21, wherein the ruminant is a bovine.

23. A method of producing the intravaginal formulation according to claim 1, comprising:
a) admixing the GnRH or GnRH analog with the carboxylic acid;
b) grinding the GnRH or GnRH analog and the carboxylic acid;
c) dispersing said ground GnRH or GnRH analog and said ground carboxylic acid in the oleaginous base.

24. An intravaginal formulation produced according to the method of claim 23.
